(19) United States
(12) Patent Application Publication
(10) Pub. No.: US 2007/0243150 A1
(43) Pub. Date: Oct. 18, 2007

(54) Methods and products for plumping lips of human beings

(76) Inventors: Pierre Bottiglieri, Conthey-Sion (CH); Candace R. Keefe, Irvine, CA (US); Jason W. Carmichael, San Clemente, CA (US)

Correspondence Address: KENNETH J. HOVET;NORDMAN, CORMANY, HAIR & COMPTON LLP, P.O. BOX 9100, 1000 TOWN CENTER DRIVE, OXNARD, CA, 93031-9100, US

(21) Appl. No.: 11/406,744

(22) Filed: Apr. 18, 2006

(51) Int. Cl. A61K 8/97; A61K 8/73

(52) U.S. Cl. 424/63; 424/70.13; 424/74

(57) ABSTRACT

Methods for plumping human comprise applying topically a first composition that includes one or more blood circulation enhancing substances, and then applying a lip gloss that also includes one or more of such substances.

### BACKGROUND OF THE INVENTION

Many methods and products for plumping human lips, e.g., Botox injections, have been proposed. Many are costly, and sometimes counterproductive, even dangerous. There is a need for less costly, harm-free products and methods for plumping human lips.

### SUMMARY OF THE INVENTION

Products for topical application to human lips include a lip preparation composition that comprises water, one or more lubricants, one or more solvents, one or more emulsifiers, one or more preservatives, one or more conditioners, one or more thickeners, one or more anti-irritants, vitamin E, one or more blood circulation promoters, one or more antimicrobial products and flavoring. In some embodiments, the lubricants may be selected from the group consisting of cyclomethicone, and glycerin; the emulsifiers, from the group consisting of glycerol stearate SE, emulsifying wax, sorbitan stearate, and polysorbate 20; the solvents, from the group consisting of butylene glycol and ethylene glycol; the thickeners, from gums such as xanthan gum; the anti-irritants and anti-microbial products, from the group consisting of allantoin and phenoyethanol; the preservatives, from the group consisting of methylparaben and propylparaben; and the blood circulation enhancers, from the group consisting of palmitoyl oligopeptide and benzyl nicotinate. In preferred embodiments, water comprises more than 50% of this composition by volume, and the solvents, emulsifiers, and thickeners, about 25% to about 35%. The blood circulation promoters comprise about 2% to about 4% of this composition.

A second composition for topical application to human lips is a lip gloss comprising solvents and emollients such as ricinus communis (castor) seed oil, bis-diglyceryl poly-acyladipate-2 and euphorbia cerifera (candelilla) wax; thickeners such as trihydoxystearin and calcium aluminum borosilicate; lubricants such as silica; moisteners, anti-irritants and toners such as aloe barbadensis leaf extract, camellia sinensis leaf extract and panax ginseng root extract; moisturizers/skin softeners such as cucumis sativus (cucumber) fruit extract; colorants such as mica, titanium dioxide, iron oxides red 6 lake, red 7 lake, and red 27; flavoring; preservatives such as propylparaben; vitamins E and C; and blood circulation promoters such as menthone glycerin acetal and palmitoyl oligopeptide. In preferred embodiments, the solvents, emollients, and thickeners comprise at least about 80% of this composition, and the blood circulation enhancers, about 2% to about 4%, by volume.

In preferred embodiments, the first, or preparatory composition, includes the following constituents by volume:

| Ingredients | % by Volume |
|---|---|
| Water | 66.79% |
| Cyclomethicone | 8.80% |
| Butylene Glycol | 5.50% |
| Glycerin | 5.50% |
| Glyceryl Stearate SE | 4.50% |
| Emulsifying Wax | 2.20% |
| Sorbitan Stearate | 1.30% |
| Polysorbate 20 | 1.10% |
| Palmitoyl Oligopeptide | 1.00% |
| Xanthan Gum | 0.40% |
| Tocopheryl Acetate | 0.05% |
| Allantoin | 0.01% |
| Benzyl Nicotinate | 0.80% |
| Phenoxyethanol | 0.75% |
| Methylparaben | 0.20% |
| Propylparaben | 0.10% |
| Flavor | 1.00% |
| Total | 100.00% |

In preferred embodiments, the second composition, the lip gloss, includes the following constituents by volume:

| Ingredients | % by Volume |
|---|---|
| Ricinus Communis (Castor) Seed Oil | 68.25%-71.95% |
| Bis-Diglyceryl Polyacyladipate-2 | 15.00% |
| Euphorbia Cerifera (Candelilla) Wax | 4.00% |
| Calcium Aluminum Borosicilicate | 0.00%-3.00% |
| Trihydroxystearin | 1.00% |
| Palmitoyl Oligopeptide | 1.00% |
| Bumetrizole | 0.10% |
| Menthone Glycerin Acetal | 0.80% |
| Silica | 0.00%-0.10% |
| Aloe Barbadensis Leaf Extract | 0.10% |
| Camellia Sinensis Leaf Extract | 0.10% |
| Panax Ginseng Root Extract | 0.10% |
| Cucumis Sativus (Cucumber) Fruit Extract | 0.10% |
| Retinyl Palmitate | 0.05% |
| Tetrahexyldecyl Ascorbate | 0.10% |
| Tocopherol | 0.10% |
| Propylparaben | 0.20% |
| Flavor | 1.00% |
| Mica | 0.00%-5.00% |
| Titanium Dioxide | 0.10%-3.00% |
| Iron Oxides | 0.00%-2.10% |
| Red 6 Lake | 0.00%-1.00% |
| Red 7 Lake | 0.00%-2.40% |
| Red 27 | 0.00%-0.80% |
| Total | 100.00% |

What is claimed is:

1. A composition for topical application to human lips comprises: a water-based composition that includes one or more blood circulation enhancing substances.

2. The composition of claim 1 comprising:

| Ingredients | % by Volume |
|---|---|
| Water | 66.79% |
| Cyclomethicone | 8.80% |
| Butylene Glycol | 5.50% |
| Glycerin | 5.50% |
| Glyceryl Stearate SE | 4.50% |
| Emulsifying Wax | 2.20% |
| Sorbitan Stearate | 1.30% |
| Polysorbate 20 | 1.10% |
| Palmitoyl Oligopeptide | 1.00% |
| Xanthan Gum | 0.40% |
| Tocopheryl Acetate | 0.05% |
| Allantoin | 0.01% |
| Benzyl Nicotinate | 0.80% |
| Phenoxyethanol | 0.75% |
| Methylparaben | 0.20% |
| Propylparaben | 0.10% |
| Flavor | 1.00% |
| Total | 100.00% |

3. A lip gloss composition for topical application to human lips comprises organic solvents and emollients, and one or more blood circulation enhancing substances.

4. The lip gloss composition of claim 3 comprising:

| Ingredients | % by Volume |
|---|---|
| Ricinus Communis (Castor) Seed Oil | 68.25%-71.95% |
| Bis-Diglyceryl Polyacyladipate-2 | 15.00% |
| Euphorbia Cerifera (Candelilla) Wax | 4.00% |
| Calcium Aluminum Borosicilicate | 0.00%-3.00% |
| Trihydroxystearin | 1.00% |
| Palmitoyl Oligopeptide | 1.00% |
| Bumetrizole | 0.10% |
| Menthone Glycerin Acetal | 0.80% |
| Silica | 0.00%-0.10% |
| Aloe Barbadensis Leaf Extract | 0.10% |
| Camellia Sinensis Leaf Extract | 0.10% |
| Panax Ginseng Root Extract | 0.10% |
| Cucumis Sativus (Cucumber) Fruit Extract | 0.10% |
| Retinyl Palmitate | 0.05% |
| Tetrahexyldecyl Ascorbate | 0.10% |
| Tocopherol | 0.10% |
| Propylparaben | 0.20% |
| Flavor | 1.00% |
| Mica | 0.00%-5.00% |
| Titanium Dioxide | 0.10%-3.00% |
| Iron Oxides | 0.00%-2.10% |
| Red 6 Lake | 0.00%-1.00% |
| Red 7 Lake | 0.00%-2.40% |
| Red 27 | 0.00%-0.80% |
| Total | 100.00% |

5. A method for plumping human lips comprises: topically applying to human lips a composition comprising a water-based composition that includes one or more blood circulation enhancing substances, and then applying, to the same lips, a composition comprising organic solvents and emollients, and one or more blood circulation enhancing substances.

6. The method of claim 5 further comprising, selecting, as the first composition, a composition comprising

| Ingredients | % by Volume |
|---|---|
| Water | 66.79% |
| Cyclomethicone | 8.80% |
| Butylene Glycol | 5.50% |
| Glycerin | 5.50% |
| Glyceryl Stearate SE | 4.50% |
| Emulsifying Wax | 2.20% |
| Sorbitan Stearate | 1.30% |
| Polysorbate 20 | 1.10% |
| Palmitoyl Oligopeptide | 1.00% |
| Xanthan Gum | 0.40% |
| Tocopheryl Acetate | 0.05% |
| Allantoin | 0.01% |
| Benzyl Nicotinate | 0.80% |
| Phenoxyethanol | 0.75% |
| Methylparaben | 0.20% |
| Propylparaben | 0.10% |
| Flavor | 1.00% |
| Total | 100.00% |

and selecting, as the second composition, a composition comprising

| Ingredients | % by Volume |
|---|---|
| Ricinus Communis (Castor) Seed Oil | 68.25%-71.95% |
| Bis-Diglyceryl Polyacyladipate-2 | 15.00% |
| Euphorbia Cerifera (Candelilla) Wax | 4.00% |
| Calcium Aluminum Borosicilicate | 0.00%-3.00% |
| Trihydroxystearin | 1.00% |
| Palmitoyl Oligopeptide | 1.00% |
| Bumetrizole | 0.10% |
| Menthone Glycerin Acetal | 0.80% |
| Silica | 0.00%-0.10% |
| Aloe Barbadensis Leaf Extract | 0.10% |
| Camellia Sinensis Leaf Extract | 0.10% |
| Panax Ginseng Root Extract | 0.10% |
| Cucumis Sativus (Cucumber) Fruit Extract | 0.10% |
| Retinyl Palmitate | 0.05% |
| Tetrahexyldecyl Ascorbate | 0.10% |
| Tocopherol | 0.10% |
| Propylparaben | 0.20% |
| Flavor | 1.00% |
| Mica | 0.00%-5.00% |
| Titanium Dioxide | 0.10%-3.00% |
| Iron Oxides | 0.00%-2.10% |
| Red 6 Lake | 0.00%-1.00% |
| Red 7 Lake | 0.00%-2.40% |
| Red 27 | 0.00%-0.80% |
| Total | 100.00% |
